(19) United States
(12) Patent Application Publication
(10) Pub. No.: US 2011/0039909 A1
(43) Pub. Date: Feb. 17, 2011

(54) METHODS AND MATERIALS FOR REDUCING GLI2 EXPRESSION

(75) Inventors: Vladimir S. Spiegelman, Fitchburg, WI (US); Saravanan Thiyagarajan, Pikesville, MD (US)

Correspondence Address: ALLISON JOHNSON, P.A., 3500 AMERICAN BLVD. W., SUITE 690, MINNEAPOLIS, MN, 55431, US

(73) Assignee: WISCONSIN ALUMNI RESEARCH FOUNDATION, Madison, WI (US)

(21) Appl. No.: 12/578,304

(22) Filed: Oct. 13, 2009

Related U.S. Application Data
(60) Provisional application No. 61/105,571, filed on Oct. 15, 2008.

(51) Int. Cl. A61K 31/7088; C07H 21/02; C12N 15/63; A61P 35/04

(52) U.S. Cl. 514/44 A; 536/23.1; 435/320.1; 514/44 R; 536/24.5

(57) ABSTRACT

Methods and materials for reducing expression of GLI2 are disclosed including nucleic acid molecules such as short hairpin RNAs that direct cleavage of GLI2 encoding transcripts and the use of such molecules for reducing prostate cancer cell growth.

### CROSS REFERENCE TO RELATED APPLICATIONS

This application claims the benefit of U.S. Provisional Application Ser. No. 60/105,571, filed Oct. 15, 2008, and incorporated herein.

### STATEMENT REGARDING FEDERALLY SPONSORED RESEARCH OR DEVELOPMENT

Funding for the work described herein was provided in part by the federal government under grant number 1R01CA121851, awarded by the National Cancer Institute. The federal government has certain rights in the invention.

### TECHNICAL FIELD

This document relates to methods and materials for reducing GLI2 expression and more particularly, to nucleic acid molecules that direct cleavage of GLI2-encoding RNA transcripts and using such nucleic acid molecules for reducing GLI2 expression.

### BACKGROUND

The glioma-associated (GLI) proteins are a group of three related proteins involved in transcriptional activation of the target genes of the Hedgehog (Hh) pathway in mammals. The GLI proteins share several regions with sequence homology, including a centrally located DNA-binding domain with five C2-H2 zinc fingers and a C-terminal transcription activation domain. Of the GLI proteins, GLI3 acts primarily as a transcriptional repressor, whereas GLI2 is the primary activator of Hh signaling. GLI1 is a transcriptional target of GLI2 and its up-regulation in response to the activation of the Hh signaling pathway depends on GLI2 protein stabilization. Mutant Gli2 proteins have been used to study various aspects of the Hh pathway. See, e.g., Bai et al., Development 129: 4753-61 (2002).

### SUMMARY

This document is based on the discovery that reduction of the levels of GLI2 protein in prostate tumor cells results in down-regulation of the Hh signaling pathway, followed by inhibition of colony formation, anchorage-independent growth, and growth of xenografts in vivo. As such, decreasing levels of GLI2 can aid in the treatment of cancer and inhibit tumor cell growth (e.g., prostate cancer). In some embodiments, methods of treating cancer can include decreasing levels of GLI2 and administering a chemotherapeutic agent as targeting GLI2 does not induce apoptosis.

In one aspect, this document features a nucleic acid molecule (e.g., ribonucleic acid, RNA) that includes or consists of first and second regions that are each from 15 to 30 nucleotides in length, where the first region includes the Gli2 target nucleotide sequence set forth in GATCTGGACAGGGATGACT (SEQ ID NO:5), and the second region has sufficient complementarity to the target nucleotide sequence for the nucleic acid molecule to direct cleavage of a GLI2-encoding RNA transcript via RNA interference.

In some embodiments, the nucleic acid molecule can be a short hairpin RNA (shRNA) where the first and second regions occur within a single strand of RNA. A linker region (e.g., from 6 to 9 nucleotides) can link the first and second regions.

In some embodiments, the nucleic acid molecule can be an intermolecular duplex, where the first region occurs within a first strand of RNA and the second region occurs within a second strand of RNA. The first and second regions of RNA each further can include two 2′ deoxyribonucleotides at their 3′-ends.

In another aspect, this document features a nucleic acid construct that includes a promoter operably linked to a nucleic acid molecule. The nucleic acid molecule includes first and second regions that are each from 15 to 30 nucleotides in length, the first region including the Gli2 target nucleotide sequence set forth in SEQ ID NO:5, and the second region having sufficient complementarity to the target nucleotide sequence for the nucleic acid molecule to direct cleavage of a GLI2-encoding RNA transcript via RNA interference.

This document also features a nucleic acid molecule (e.g., RNA) that includes or consists of first and second regions that are each from 15 to 30 nucleotides in length. The first region corresponding to a target nucleotide sequence in the human Gli2 mRNA coding sequence set forth in SEQ ID NO: 1; and the second region having sufficient complementarity to the target nucleotide sequence for the nucleic acid molecule to direct cleavage of a GLI2-encoding RNA transcript via RNA interference, wherein the first and second regions occur within a single strand of the nucleic acid molecule and are linked by a linker region. The first region can include from 19 to 30 consecutive nucleotides of SEQ ID NO: 1. For example, the target nucleotide sequence can have the nucleotide sequence set forth in SEQ ID NO: 3, 4, 5, 6, or 7. In some embodiments, the nucleic acid molecule is a shRNA. The linker region can include from 6 to 9 nucleotides.

In another aspect, this document features a nucleic acid construct that includes a promoter operably linked to a nucleic acid molecule, the nucleic acid molecule including first and second regions that are each from 15 to 30 nucleotides in length, the first region corresponding to a target nucleotide sequence in the human Gli2 mRNA coding sequence set forth in SEQ ID NO: 1; and the second region having sufficient complementarity to the target nucleotide sequence for the nucleic acid molecule to direct cleavage of a GLI2-encoding RNA transcript via RNA interference, wherein the first and second regions occur within a single strand of the nucleic acid molecule and are linked by a linker region. The promoter can be a HII promoter.

This document also features a composition that includes a nucleic acid molecule described herein and a pharmaceutically acceptable excipient. The composition decreases the level of a Gli2 mRNA or polypeptide when introduced into a cell.

In yet another aspect, this document features a method of inhibiting tumor cell growth (e.g., prostate tumor cell growth) in an individual. The method includes administering to an individual a composition that inhibits expression of Gli2 by RNA interference. The composition that inhibits expression of Gli2 can include a nucleic acid molecule described herein and a pharmaceutically acceptable excipient. For example, the nucleic acid molecule can include first and second regions that are each from 15 to 30 nucleotides in length, the first region including the Gli2 target nucleotide sequence set forth in SEQ ID NO:5, and the second region having sufficient complementarity to the target nucleotide sequence for the nucleic acid molecule to direct cleavage of a GLI2-encoding RNA transcript via RNA interference.

This document also features a method of treating cancer. The method can include administering to an individual a composition that inhibits expression of Gli2. The method further can include administering a chemotherapeutic agent (e.g. a chemotherapeutic agent capable of causing apoptosis of cancer cells). The cancer can be selected from the group consisting of prostate cancer, basal cell carcinoma, medulloblastoma, pancreatic cancer, hepatocellular carcinoma, gastric cancer, breast cancer, and lung cancer. The composition that inhibits expression of Gli2 can include a nucleic acid molecule described herein and a pharmaceutically acceptable excipient.

Unless otherwise defined, all technical and scientific terms used herein have the same meaning as commonly understood by one of ordinary skill in the art to which this application pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is the nucleotide sequence of the human Gli2 mRNA from GenBank Accession No. NM_{—}005270.4. Nucleotides 31 to 4791 encode the human GLI2 protein.

FIG. 2 is the amino acid sequence of the human GLI2 protein from GenBank Accession No. NP_{—}005261.

FIG. 3A is a schematic of a DNA template encoding a shRNA.

FIG. 3B contains five examples of DNA templates encoding shRNAs. Target sequences 1-5 are SEQ ID NOs:3-7, respectively.

FIG. 4 is a photograph of a Western analysis for examining the effect of shGli2 constructs on GLI2 protein levels. Lane 1 represents 293T cells transfected with control shRNA; Lane 2 is 293T cells transfected with vector pLVTHM; Lanes 3 to 7 represent 293T cells transfected with shRNA constructs targeted to SEQ ID NOs:3-7, respectively.

FIGS. 5A-5D depict that shRNA-mediated inhibition of GLI2 inhibits GLI-dependent transcription in prostate cancer cells.

FIG. 5A is an immunoblot analysis of GLI2 protein isolated from 293T cells transfected with shRNA plasmids as indicated. Data in FIG. 5A are representative of two independent experiments.

FIGS. 5B-5D are graphs depicting the amount of light produced in prostate cancer cells transfected with pGL3-Bcl2promo (FIG. 5B), K17-luc (FIG. 5C), 8×3′Gli BS-LucII (FIG. 5D), pSV4013-gal (Promega), and shRNA expression plasmids as indicated. Luciferase activity was estimated using luciferase reporter assay reagent (Promega), β-galactosidase was used for normalization and estimated using β-galactosidase assay reagent (Pierce). *, P<0.01, compared with cells transfected with scrambled shRNA (Student's t test).

FIGS. 6A-6C depict suppression of proliferation and anchorage-independent growth of prostate cancer cells on knockdown of GLI2 protein expression.

FIG. 6A is a graph showing the number of colonies formed per plate in RWPE1 cells (control) and various prostate cancer cell lines transfected with scrambled and GLI2 shRNA. Transfected cells were selected with puromycin (Sigma), allowed to form colonies, fixed with 10% formalin, stained with 2% Gentian violet (Ricca Chem. Co.) and analyzed statistically.

FIGS. 6B and 6C depict anchorage-independent colony formation for 22Rv1 cells infected with lentiviral constructs encoding either scrambled shRNA or GLI2 shRNA. Colony formation was determined under low magnification (10×) using an inverted microscope (FIG. 6B) and colonies were counted (FIG. 6C). In these figures, represents P<0.01 (as determined by the Student's t test), compared with cells transfected with scrambled shRNA.

FIGS. 7A-7C depict the inhibition of growth of 22Rv1 xenografts by down-regulation of GLI2.

FIG. 7A depicts linear regression analysis of tumor growth in nude mice injected with 22Rv1 cells expressing either GLI2 shRNA or scrambled shRNA (control). The linear regression analysis shows the rate of mean tumor or carcinoma area growth and tumor multiplicity as a function of time using S-plus Software (Insightful).

FIGS. 7B and 7C depict a Kaplan-Meier survival analysis (i.e., average time to a target tumor volume of 500 mm³) for xenografts from nude mice injected with 22Rv1 cells expressing either GLI2 shRNA or scrambled shRNA (FIG. 7B), and statistical analysis of the differences between xenografts from nude mice injected with 22Rv1 cells expressing GLI2 shRNA or scrambled shRNA according to log-rank (FIG. 7C). In these figures, * represents P<0.05 (as determined by the Student's t test), compared with cells transfected with scrambled shRNA.

FIGS. 8A-8B depict the effect of overexpression of Gli2 on proliferation of RWPE1 cells.

FIG. 8A is a graph showing the results of a cell proliferation assay using RWPE1 cells stably transfected with pcDNA3.1-Flag-Gli2 or pcDNA3.1 (control). Proliferation was estimated with CellTiter 96 AQueous One Solution Reagent (Promega) at 24, 48 and 72 hours after an initial absorbance reading at 490 nm using an ELISA reader. The levels of GLI2 in the stably transfected RWPE1 cells were analyzed by immunoblotting with the Gli2 antibody (inset).

FIG. 8B shows two graphs depicting the flow analysis results for cell cycle analysis carried out in RWPE1 cells stably transfected with pcDNA3.1-Flag-Gli2 or pcDNA3.1 (control). Harvested cells were fixed with 95% ethanol, treated with RNase A and stained with propidium iodide for 30 minutes at 37° C. and analyzed by flow cytometry.

### DETAILED DESCRIPTION

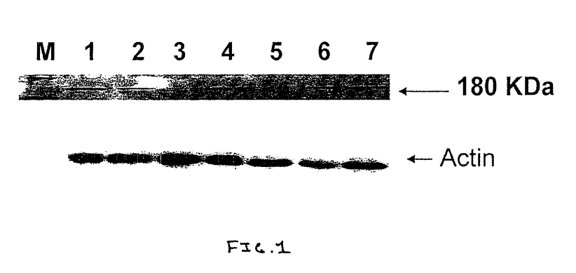

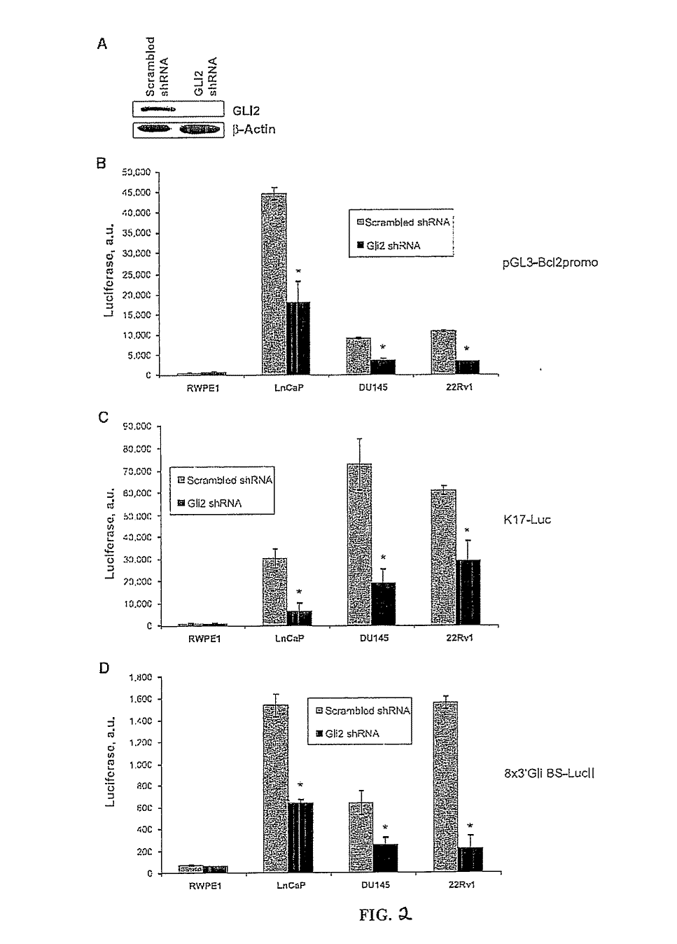

This document is based on the discovery that reduction of the levels of GLI2 protein in prostate tumor cells results in down-regulation of the Hh signaling pathway, followed by inhibition of colony formation, anchorage-independent growth, and growth of xenografts in vivo. Decreasing levels of GLI2 can aid in the treatment of cancer and inhibit tumor cell growth. The term “GLI2” as used herein refers to mammalian glioma-associated family member GLI2 (e.g., from mice or humans). The nucleic acid sequence encoding human GLI2 can be found in GenBank under Accession No. NM_{—}005270.4 and is provided in FIG. 1 (SEQ ID NO:1). The amino acid sequence of human GLI2 protein can be found in GenBank under Accession No. NP_{—}005261 and is provided in FIG. 2 (SEQ ID NO:2). GLI2 protein is expressed at very low levels in normal prostate epithelial cells but is overexpressed in prostate cancer cells.

### Decreasing Concentration of GLI2

This document provides nucleic acid molecules that direct cleavage of a GLI2-encoding RNA transcript, or otherwise result in reductions in the concentration of GLI2, and isolated mammalian cells (e.g., human cells) that include such nucleic acid molecules. Such nucleic acid molecules can be used to reduce levels of GLI2 in cells, inhibit tumor cell growth, and treat cancers, including solid cancers such as medulloblastoma, lung, breast, pancreatic, gastric, hepatocellular carcinoma, and prostate as well as skin cancers such as basal cell carcinoma. Nucleic acid molecules described herein are particularly useful for inhibiting prostate cancer cell growth and treating prostate cancer.

As used herein, the phrase “nucleic acids that direct cleavage of a GLI2-encoding RNA transcript” means any nucleic acid molecule or nucleic acid molecule analog that results in the reduction of the level of mRNA or pre-mRNA encoding the GLI2 protein inside a cell. The phrase “GLI2-encoding RNA transcript” includes any naturally occurring polymorphic variants of RNA transcripts encoding GLI2. Such nucleic acid molecules can be RNAs that act by inducing RNA interference (RNAi), i.e., the double-stranded, RNA-directed degradation of endogenous transcripts of corresponding sequence. For example, such nucleic acid molecules can be small interfering RNAs (siRNAs), or small hairpin RNAs (shRNAs) that can be processed into siRNAs within cells, that induce RNAi-mediated degradation of transcripts encoding GLI2. Alternatively, such nucleic acid molecules can be enzymatic nucleic acids, either RNA molecules, DNA molecules, or analogs thereof, that directly cleave RNA transcripts encoding GLI2 (e.g., ribozymes or DNAzymes). In other embodiments, such nucleic acid molecules can be antisense oligonucleotides that specifically hybridize with mRNA or pre-mRNA encoding GLI2 and promote the cleavage and degradation of these transcripts by cellular endonucleases, such as ribonuclease-H (RNase-H).

As used herein, the term “nucleic acid” refers to an oligomer or polymer of ribonucleic acid (RNA) or deoxyribonucleic acid (DNA) or analogs thereof. Nucleic acid analogs can be modified at the base moiety, sugar moiety, or phosphate backbone to improve, for example, stability, hybridization, or solubility of a nucleic acid. Modifications at the base moiety include substitution of modified purine or pyrimidine bases. For example, deoxyuridine for deoxythymidine, and 5-methyl-2′-deoxycytidine and 5-bromo-2′-deoxycytidine for deoxycytidine. Other examples of nucleobases that can be substituted for a natural base include 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Other useful nucleobases include those disclosed, for example, in U.S. Pat. No. 3,687,808.

Modifications of the sugar moiety can include modification of the 2′ hydroxyl of the ribose sugar to form 2′-O-methyl or 2′-O-allyl sugars. For example, 2′-β-methoxyethyl sugar moieties can be substituted. The deoxyribose phosphate backbone can be modified to produce morpholino nucleic acids, in which each base moiety is linked to a six-membered, morpholino ring, or peptide nucleic acids, in which the deoxyphosphate backbone is replaced by a pseudopeptide backbone (e.g., an aminoethylglycine backbone) and the four bases are retained. See, for example, Summerton and Weller (1997) Antisense Nucleic Acid Drug Dev. 7:187-195; and Hyrup et al. (1996) Bioorgan. Med. Chem. 4:5-23. In addition, the deoxyphosphate backbone can be replaced with, for example, a phosphorothioate or phosphorodithioate backbone, a phosphoroamidite, chiral phosphorothioates, alkyl phosphotriester, aminoalkylphosphotriester, alkyl phosphonate, thionoalkylphosphonate, phosphinate, phosphoramidate, thionophosphoramidate, thionoalkylphosphotriester, or boranophosphate backbone, and various salt forms thereof. Other examples of modified backbones include siloxane, sulfide, sulfoxide, sulfone, sulfonate, sulfonamide, and sulfamate backbones; formacetyl and thioformacetyl backbones; alkene-containing backbones; methyleneimino and methylenehydrazino backbones; and amide backbones. See, for example, U.S. Pat. Nos. 4,469,863, 5,235,033, 5,750,666, and 5,596,086 for methods of preparing nucleic acids with modified backbones.

Nucleic acid molecules described herein also can be modified by chemical linkage to one or more moieties or conjugates that enhance the activity, cellular distribution or cellular uptake of the nucleic acid. Such moieties include but are not limited to lipid moieties (e.g., a cholesterol moiety); cholic acid; a thioether moiety (e.g., hexyl-5-tritylthiol); a thiocholesterol moiety; an aliphatic chain (e.g., dodecandiol or undecyl residues); a phospholipid moiety (e.g., di-hexadecyl-rac-glycerol or triethyl-ammonium 1,2-di-O-hexadecyl-rac-glycero-3-H-phosphonate); a polyamine or a polyethylene glycol (PEG) chain; adamantane acetic acid; a palmityl moiety; or an octadecylamine or hexylamino-carbonyl-oxycholesterol moiety. The preparation of such nucleic acid conjugates is disclosed in, for example, U.S. Pat. Nos. 5,218,105 and 5,214,136.

Confirmation that a nucleic acid molecule induces degradation of RNA transcripts encoding GLI2 can be obtained by demonstrating a quantitative reduction in transcripts encoding GLI2, or a reduction in GLI2 protein itself, using any method known in the art. One method for measuring GLI2-encoding transcripts includes real-time quantitative RT-PCR, as described by Winer et al. (Anal. Biochem. 270:41-49 (1999)). Western Blot analysis (e.g., quantitative Western blot analysis) as described by Gingrich et al., (BioTechniques 29:636-642 (2000)) can be used to demonstrate a reduction in GLI2 protein. The baseline level of GLI2-encoding RNA transcripts or GLI2 protein can be determined in control experiments before, or in the absence of, treatment by a nucleic acid molecule described herein.

In some embodiments, methods of quantitating mRNA or protein levels can include normalizing the results for differences in the amount of total RNA or total protein in the sample to be quantitated. Generally, normalization can be achieved by quantitating an internal standard such as the product of a ubiquitously expressed “housekeeping” gene. For example, quantitative RT-PCR assays can be normalized by simultaneously quantitating mRNA encoding glyceraldehyde-3-phosphate dehydrogenase (GAPDH), which is expressed at generally invariant levels in different cell types under different treatment conditions (Winer et al., Anal. Biochem. 270:41-49 (1999)). Differences in the levels of GAPDH mRNA are representative of different amounts of input mRNA templates between reactions, and can be used as an internal standard by which to adjust levels of the quantitated transcript, to adjust for such differences in input mRNA templates. Similarly, the levels of GAPDH protein can be used to normalize for differences in the amounts of input total protein during protein quantitation assays.

In general, siRNAs are short intermolecular duplexes composed of two distinct strands of RNA (i.e., sense and antisense strands), each approximately 21 nucleotides in length. The two strands are partly complementary and hybridize to form approximately 19 base-pairs, with single-stranded 3′ overhangs of 1-3 nucleotides (e.g., 2 nucleotides). The base-paired region of siRNAs generally corresponds substantially to a “target sequence” and its complement in the GLI2-encoding RNA transcript targeted for degradation by the RNAi process and cellular machinery. In some embodiments, the base-paired region of siRNA corresponds exactly to the target sequence and its complement in the GLI2-encoding RNA transcript. The sequence of the overhangs makes only a small contribution to the overall specificity of target recognition, but the identity of the nucleotide adjacent to the paired region can have an effect. The 3′ overhangs can be composed of either ribonucleotides or 2′-deoxyribonucleotides with no apparent differences in efficacy. In some embodiments, siRNAs with 2′-deoxyribonucleotide overhangs can be more resistant to certain cellular nucleases. See, for example, Tuschl et al., Genes Dev. 13:3191-3197 (1999) and Elbashir et al., EMBO J. 20:6877-6888 (2001) for the design of effective siRNA molecules.

Target sequences in targeted RNA transcripts preferably have the sequence AA(19N)UU, where N is any contiguous 19 nucleotides. Target sequences are chosen from the sequences present in mature mRNAs, but can reside in either coding or non-coding regions. Particularly useful target sequences are readily accessible to the siRNA, i.e., not involved in a stable base-paired structure within the mature transcript, and not specifically bound by an RNA-binding protein. RNA folding algorithms, such as the “Sfold” algorithm available through an Sfold web-server developed by Ding, Chan and Lawrence (described in Nucleic Acids Res. 32 (Web Server issue):W135-41 (2004); see world wide web at sfold.wadsworth.org) can be used for picking target sequences that have a greater likelihood of being accessible.

The individual single-stranded RNAs that include siRNAs can be synthesized outside of cells (exogenously) or within cells (endogenously). The two complementary single strands can anneal to form an intermolecular RNA duplex, i.e., the siRNA. The annealing step also can occur exogenously or endogenously. Exogenously synthesized single-stranded RNAs can be synthesized chemically using conventional RNA synthesis methods. For example, the nucleic acid molecules can be chemically synthesized using appropriately protected ribonucleoside phosphoramidites and a conventional DNA/RNA synthesizer. Various methods for RNA synthesis are disclosed in, e.g., Usman et al., J. Am. Chem. Soc., 109:7845-7854 (1987) and Scaringe et al., Nucleic Acids Res., 18:5433-5441 (1990). Custom and large-scale siRNA synthesis services are available from commercial vendors such as Ambion (Austin, Tex., USA), Dharmacon Research (Lafayette, Colo., USA), Pierce Chemical (Rockford, Ill., USA), ChemGenes (Ashland, Mass., USA), Proligo (Hamburg, Germany), and Cruachem (Glasgow, UK). Nucleic acid molecules also can be synthesized enzymatically using an RNA polymerase and a DNA construct containing an appropriate promoter sequence operably linked to the template sequence. Exogenously synthesized single-stranded RNAs can be purified before annealing to form siRNA duplexes. Endogenously synthesized single-stranded RNAs can be synthesized by cellular RNA polymerases using a DNA construct that contains an appropriate promoter sequence operably linked to the template sequence.

Small hairpin RNAs, or shRNAs, are single-stranded RNAs having two regions connected by a linker region. The first region of a shRNA corresponds to a target nucleotide sequence in the GLI2-encoding RNA and the second region has sufficient complementarity to the first region such that regions can pair with one another, allowing the single strand to fold into an intramolecular duplex with a stem-loop type structure and direct cleavage of a GLI2-encoding RNA transcript via RNA interference. The linker region forms the “loop” in the stem-loop structure when the first and second regions pair with each other. Typically, the linker is from 3 to 9 nucleotides (e.g., 5 to 10, 6 to 9, 6, 7, 8, or 9 nucleotides) in length. While the sequence of the loop is not generally important, there are some general guidelines for choosing the loop sequence. Typically, the loop sequence is not related to sequences adjacent to or within the target sequence and palindromic sequences are avoided. The stem region of the shRNA typically is 15 to 30 nucleotides (e.g., 15 to 20, 19) in length, and the 3′ end of the shRNA extending beyond the paired region typically is composed of multiple uracil residues. The base-paired regions of shRNAs can correspond exactly with the target sequence or have sufficient complementarity for the nucleic acid molecule to direct cleavage of a GLI2-encoding RNA transcript via RNA interference.

The shRNAs presented in FIG. 3 have been specifically designed to target RNA transcripts encoding GLI2, and to not target transcripts encoding other GLI proteins such as GLI1 and GLI3. As shown in Example 1, shRNAs targeting SEQ ID NO:3 (GGAAGGTACCATTACGAGC) and SEQ ID NO:5 (GATCTGGACAGGGATGACT) in GLI2-encoding RNA resulted in a decrease in GLI2 expression when introduced by lipofection into 293T cells in culture. The shRNA targeted to the sequence set forth in SEQ ID NO:5 was particularly useful.

Besides the shRNA compounds provided in FIG. 3, additional compounds targeted to different sites within RNA transcripts encoding GLI2 can be designed and synthesized according to general guidelines provided herein and generally known to skilled artisans. See e.g., Elbashir, et al. (Nature 411: 494-498 (2001)).

Like the individual strands of siRNAs, shRNAs can be synthesized either endogenously or exogenously. Endogenously synthesized shRNAs can be synthesized by cellular RNA polymerases using a nucleic acid construct that contains an appropriate promoter sequence operably linked to a DNA template (e.g., a nucleic acid molecule having first and second regions as described above; see also FIG. 3). Exogenously synthesized shRNAs can be synthesized chemically, for example, using phosphoramidite chemistry, or can be synthesized enzymatically, using an RNA polymerase and a nucleic acid construct containing an appropriate promoter sequence operably linked to the template sequence. Exogenously synthesized shRNAs can be purified before being used to induce RNAi and the degradation of a GLI2 encoding RNA transcript.

### Enzymatic Nucleic Acids

The term “enzymatic nucleic acid molecules” or “enzymatic nucleic acids” as used herein refers to a nucleic acid molecule that has complementarity to a target in a GLI2 encoding RNA, and also has ability to enzymatically cleave the target RNA specifically. That is, the enzymatic nucleic acid molecule is able to intermolecularly cleave target RNA and thereby result in the degradation of the target RNA molecule. The complementary regions of the enzymatic nucleic acid allow sufficient hybridization of the enzymatic nucleic acid molecule to the target RNA and thus permit preferential cleavage of the target RNA. Typically, the enzymatic nucleic acid molecule has at least 50% complementarity (e.g. from at least 50% to at least 75%, at least 80%, at least 90%, at least 95%, at least 99%, or even 100% complementarity) with the target. See, for example, Werner and Uhlenbeck, Nucleic Acids Res. 23:2092-2096 (1995); Hammann et al., Antisense Nucleic Acid Drug Dev., 9:25-31 (1999)). Enzymatic nucleic acids can be modified at the base, sugar, and/or phosphate groups to enhance stability within host cells, or improve catalytic activity as described above. Non-limiting examples of enzymatic nucleic acids include ribozymes, catalytic RNAs, enzymatic RNAs, catalytic DNAs, aptazymes or aptamer-binding ribozymes, regulatable ribozymes, catalytic oligonucleotides, nucleozymes, DNAzymes, RNA enzymes, endoribonucleases, endonucleases, minizymes, leadzymes, oligozymes, or DNA enzymes. All of these terms describe specific types of nucleic acid molecules with catalytic activity.

In general, enzymatic nucleic acids with RNA endonuclease activity act by first binding to a target RNA. Such binding occurs through the target-binding portion of the enzymatic nucleic acid, which is held in close proximity to an enzymatic portion of the molecule, which acts to cleave the target RNA. Thus, the enzymatic nucleic acid, for example, first recognizes and then binds a target RNA through complementary base pairing, and once bound to the correct site, acts to enzymatically cleave the target RNA. Strategic cleavage of such a target RNA will lead to the destabilization and degradation of the target RNA, or otherwise destroy its ability to direct synthesis of an encoded protein. After an enzymatic nucleic acid has bound and cleaved its RNA target, the cleaved target RNA is released from the enzymatic nucleic acid, so that the enzymatic nucleic acid is freed to search for, and cleave another target, thereby repeatedly binding and cleaving multiple target RNAs. In addition, the enzymatic nucleic acid is a highly specific inhibitor of gene expression, with the specificity of inhibition depending not only on the base-pairing mechanism of binding to the target RNA, but also on the mechanism of target RNA cleavage. Single mismatches, or base-substitutions, near the site of cleavage can completely eliminate catalytic activity of an enzymatic nucleic acid molecule.

As with siRNAs and shRNAs, the choice of an appropriate target sequence is important for the function of GLI2-directed enzymatic nucleic acids, and the accessibility of a target sequence is a factor for the efficient cleavage of a specific RNA transcript by a corresponding ribozyme. Preferably the target sequence chosen is readily accessible to the enzymatic nucleic acid, i.e., not involved in a stable base-paired structure within the mature transcript, and not specifically bound by an RNA-binding protein. RNA folding algorithms, such as the “Sfold” algorithm described above can be useful for picking target sequences that have a greater likelihood of being accessible.

Ribozymes can be designed to specifically pair with virtually any target RNA and cleave the phosphodiester backbone at a specific location, thereby functionally inactivating the GLI2 encoding RNA and preventing expression of GLI2 protein. Hammerhead ribozymes are useful for destroying particular mRNAs, although various ribozymes that cleave mRNA at site-specific recognition sequences can be used. Hammerhead ribozymes cleave mRNAs at locations dictated by flanking regions that form complementary base pairs with the target mRNA. The sole requirement is that the target RNA contains a 5′-UG-3′ nucleotide sequence. The construction and production of hammerhead ribozymes is known in the art. See, for example, U.S. Pat. No. 5,254,678 and WO 02/46449 and references cited therein. Hammerhead ribozyme sequences can be embedded in a stable RNA such as a transfer RNA (tRNA) to increase cleavage efficiency in vivo. Perriman, et al., Proc. Natl. Acad. Sci. USA, 92(13):6175-6179 (1995); de Feyter and Gaudron, METHODS IN MOLECULAR BIOLOGY, Vol. 74, Chapter 43, “Expressing Ribozymes in Plants”, Edited by Turner, P. C, Humana Press Inc., Totowa, N.J. RNA endoribonucleases such as the one that occurs naturally in Tetrahymena thermophila, and which have been described extensively by Cech and collaborators can be useful. See, for example, U.S. Pat. No. 4,987,071.

### Antisense Oligonucleotides

Antisense oligonucleotides are nucleic acid molecules that can be used to decrease levels of GLI2 protein. The antisense oligonucleotides in accordance with this document are at least 8 nucleotides in length. For example, antisense oligonucleotides can be about 8, about 9, from about 10 to about 20 (e.g., 11, 12, 13, 14, 15, 16, 17, 18, 19, or 20 nucleotides in length), from 15 to 20, from 18 to 25, or even from 20 to 50 nucleotides in length. In other embodiments, antisense oligonucleotides can be used that are greater than 50 nucleotides in length, including the full-length sequence of a GLI2 mRNA. Antisense oligonucleotides can be modified at the base moiety, sugar moiety, or phosphate backbone to improve, for example, stability, hybridization, or solubility of a nucleic acid, as described above.

Methods for synthesizing antisense oligonucleotides are known, including solid phase synthesis techniques. Equipment for such synthesis is commercially available from several vendors including, for example, Applied Biosystems (Foster City, Calif.). Alternatively, expression vectors that contain a regulatory element that directs production of an antisense transcript can be used to produce antisense oligonucleotides.

Antisense oligonucleotides can bind to a nucleic acid encoding GLI2, including DNA encoding GLI2 RNA (including pre-mRNA and mRNA) transcribed from such DNA, and also cDNA derived from such RNA, under physiological conditions (i.e., physiological pH and ionic strength). For example, an antisense oligonucleotide can hybridize under physiological conditions to the nucleotide sequence set forth in FIG. 1. Typically, antisense molecules are complementary to a target sequence along a single contiguous sequence of the sense molecule. However, in certain embodiments, an antisense molecule can bind to substrate such that the substrate molecule forms a loop, and/or an antisense molecule can bind such that the antisense molecule itself forms a loop. Thus, the antisense molecule can be complementary to two (or even more) non-contiguous substrate sequences, or two (or even more) non-contiguous sequence portions of an antisense molecule can be complementary to a target sequence or both.

It is understood in the art that the sequence of an antisense oligonucleotide need not be 100% complementary to that of its target nucleic acid to be hybridizable under physiological conditions. Antisense oligonucleotides hybridize under physiological conditions when binding of the oligonucleotide to the GLI2 nucleic acid interferes with the normal function of the GLI2 nucleic acid and non-specific binding to non-target sequences is minimal.

Target sites for GLI2 antisense oligonucleotides include the regions encompassing the translation initiation or termination codon of the open reading frame (ORF) of the gene. In addition, the ORF has been targeted effectively in antisense technology, as have the 5′ and 3′ untranslated regions. Furthermore, antisense oligonucleotides have been successfully directed at intron regions and intron-exon junction regions. Further criteria can be applied to the design of antisense oligonucleotides. Such criteria are well known in the art, and are widely used, for example, in the design of oligonucleotide primers. These criteria include the lack of predicted secondary structure of a potential antisense oligonucleotide, an appropriate G and C nucleotide content (e.g., approximately 50%), and the absence of sequence motifs such as single nucleotide repeats (e.g., GGGG runs). The effectiveness of antisense oligonucleotides at modulating expression of a GLI2 nucleic acid can be evaluated by measuring levels of the GLI2 mRNA or protein (e.g., by Northern blotting, RT-PCR, Western blotting, ELISA, or immunohistochemical staining).

Pharmaceutical Compositions and Formulations

Nucleic acid molecules described herein can be formulated as pharmaceutical compositions. Typically, a pharmaceutical composition includes a nucleic acid molecule described herein and a physiologically acceptable carrier. For oral delivery, a nucleic acid molecule can be incorporated into a formulation that includes pharmaceutically acceptable carriers such as binders (e.g., gelatin, cellulose, gum tragacanth), excipients (e.g., starch, lactose), lubricants (e.g., magnesium stearate, silicon dioxide), disintegrating agents (e.g., alginate, Primogel, and corn starch), sweetening or flavoring agents (e.g., glucose, sucrose, saccharin, methyl salicylate, and peppermint), or combinations thereof. The formulation can be orally delivered in the form of enclosed gelatin capsules or compressed tablets. Capsules and tablets can be prepared by any conventional technique. Capsules and tablets also can be coated with various coatings known in the art to modify the flavors, tastes, colors, and shapes of the capsules and tablets. In addition, liquid carriers such as oils can be included in capsules. Suitable oral formulations also can be in the form of suspension, syrup, chewing gum, wafer, elixir, and the like. If desired, conventional agents for modifying flavors, tastes, colors, and shapes of the special forms also can be included. In addition, for convenient administration by enteral feeding tube in patients unable to swallow, the nucleic acid molecules can be dissolved in an acceptable lipophilic vegetable oil vehicle such as olive oil, corn oil and safflower oil.

For parenteral delivery, nucleic acid molecules can be formulated as a solution or suspension, or in a lyophilized form capable of conversion into a solution or suspension form before use. In such formulations, diluents or pharmaceutically acceptable carriers such as sterile water and physiological saline buffer can be used. Other conventional solvents, pH buffers, stabilizers, anti-bacterial agents, surfactants, antioxidants, or combinations thereof also can be included. For example, useful diluents or pharmaceutically acceptable carriers can include sodium chloride, acetate, citrate or phosphate buffers, glycerin, dextrose, fixed oils, methyl parabens, polyethylene glycol, propylene glycol, sodium bisulfate, benzyl alcohol, ascorbic acid, and the like. Parenteral formulations can be stored in any conventional containers such as vials and ampules.

For topical administration (e.g., nasal, bucal, mucosal, rectal, or vaginal applications), the active compounds can be formulated into lotions, creams, ointments, gels, powders, pastes, sprays, suspensions, drops and aerosols. Thus, one or more thickening agents, humectants, and stabilizing agents can be included in the formulations. Examples of such agents include, but are not limited to, polyethylene glycol, sorbitol, xanthan gum, petrolatum, beeswax, or mineral oil, lanolin, squalene, and the like. A special form of topical administration can be delivery by a transdermal patch. Methods for preparing transdermal patches are disclosed, e.g., in Brown, et al., Annual Review of Medicine, 39:221-229 (1988).

Subcutaneous implantation for sustained release of the nucleic acid molecules also may be a suitable route of administration. This entails surgical procedures for implanting an active compound in any suitable formulation into a subcutaneous space, e.g., beneath the anterior abdominal wall. Hydrogels can be used as a carrier for the sustained release of the active compounds. Hydrogels are known in the art and are typically made by crosslinking high molecular weight biocompatible polymers into a network that swells in water to form a gel like material. Biodegradable or biosorbable hydrogels can be used. Hydrogels made of polyethylene glycols, collagen, or poly(glycolic-co-L-lactic acid) may be useful. See, e.g., Phillips et al., J. Pharmaceut. Sci. 73:1718-1720 (1984).

Pharmaceutical compositions also can include one or more chemotherapeutic agents for inhibiting cancer cell growth or treatment of cancer (e.g., prostate cancer). For example, a pharmaceutical composition can include an alkylating agent such as a nitrogen mustard, ethylenimine or methylmelamine, alkyl sulfonate, nitrosourea, or triazene; an antimetabolite such as a folic acid analog, pyrimidine analog, or purine analog; a natural product such as a vinca alkaloid, epipodophyllotoxin, antibiotic, enzyme, or biological response modifier (e.g., interferon alpha or beta); platinum coordination complex; anthracendione; substituted urea; methylhydrazine derivative; adrenocortical suppressant; hormones and antagonists such as adrenocorticosteroids, progestins, estrogens, antiestrogen, androgens, antiandrogen (e.g., Flutamide or Nilutamide), gonadotropin-releasing hormone analog (e.g., Leuprolide or Goserelin), or combinations thereof.

In some embodiments, a pharmaceutical composition also can include an uptake agent that specifically enhances or increases the uptake of the nucleic acid molecules by target cells, or specifically enhances or improves the delivery of the nucleic acid molecules to the target cells. An uptake agent can include any compound that, when used in formulating the pharmaceutical composition described herein, results in a net increase in the amount of the nucleic acid molecule taken up by the target cells, such that a decrease of at least about a 5% (e.g., at least about 7%, at least about 8%, at least about 9%, at least about 10%, or even at least about 15%) in GLI2-encoding transcripts, or GLI2 protein is observed in those target cells treated with compositions including the uptake agent, as compared with cells treated with identical compositions, but lacking the uptake agent. Examples of uptake agents include, but are not limited to, amphipathic compounds and compounds used to formulate liposomes, immunoliposomes, or pegylated immunoliposomes. Examples of such compounds include Lipofectin, Lipofectamine, or Cellfectin, and various polycations and polyethylene glycols. Examples of uptake agents that improve or enhance delivery of the nucleic acids to specific target organs, tissues or cells include monoclonal antibodies or other compounds that are capable of interacting with receptors on the surface of target cells or target tissues. Typically, such agents are co-formulated with the nucleic acid molecules described herein as well as with uptake agents. See, for example, U.S. Patent Application Publication 2004/0156909.

It will be apparent that a therapeutically effective amount for each active compound to be included in a pharmaceutical composition can vary with factors including, e.g., the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and combinations thereof. The amount of administration can also be adjusted as the various factors change over time.

Methods for delivering nucleic acid molecules into specific tissues of mammal, and by specific routes, have been developed. Such methods can include formulating particular pharmaceutical compositions or modifying the nucleic acids that are to be delivered. Non-limiting examples of modifications that can be used to improve bioavailability of a nucleic acid are described in WO 98/49348, WO 2004/029075 and WO 2004/065579, U.S. Pat. Nos. 6,153,737 and 6,395,492, and in U.S. Patent Application Publications 2002/0188101 and 2003/0064492. In some embodiments, long-circulating liposomal compositions can be formulated as described in WO 99/59547. Compositions and methods of using modified oligonucleotides for topical delivery are described in WO 99/60617. Compositions and methods of using modified oligonucleotides for pulmonary delivery are described in U.S. Patent Application Publications 2003/0157030 and 2004/0063654. Compositions and methods of using modified oligonucleotides for delivery via the alimentary canal are described in WO 99/01579 and WO 99/60012, as well as in U.S. Pat. No. 6,747,014, with the latter two publications specifically teaching methods for rectal administration of therapeutic nucleic acids. In some embodiments, cationic liposomes or pegylated immunoliposomes (e.g., 85 nm pegylated immunoliposomes) studded with monoclonal antibodies selected to interact with specific cellular receptors, can be used to facilitate the uptake of nucleic acid molecules.

Methods of Treating Cancer

A nucleic acid molecule described herein can be administered to a mammal such as a human patient that has been diagnosed with cancer (e.g., medulloblastoma, lung, gastric, hepatocellular carcinoma, breast, pancreatic, prostate, or basal cell carcinoma). Suitable nucleic acid molecules direct cleavage of a GLI2-encoding RNA transcript, or otherwise result in reductions in the concentration of GLI2. Nucleic acid molecules that can be used include, for example, siRNAs, shRNAs, enzymatic nucleic acids, or antisense oligonucleotides as discussed above. Treatment of a cancer can include reducing the severity of the disease or slowing progression of the disease. Nucleic acid molecules described herein also can be administered prophylactically in patients at risk for developing cancer to prevent development of symptoms of the disease from occurring, delaying onset of symptoms, or lessening the severity of subsequently developed disease symptoms. In either case, an amount of a nucleic acid molecule effective to reduce the concentration of GLI2 is administered to the patient. As used herein, the term “effective amount” refers to an amount of a nucleic acid molecule that reduces the deleterious effects of the cancer without inducing significant toxicity to the host. Effective amounts of nucleic acid molecules can be determined by a physician, taking into account various factors that can modify the action of drugs such as overall health status, body weight, sex, diet, time and route of administration, other medications, and any other relevant clinical factors. Typically, an amount of a nucleic acid molecule is provided such that, when introduced into cells, the nucleic acid molecule results in the reduction of RNA transcripts encoding GLI2 or a reduction in GLI2 protein levels by at least 5%, at least 10%, at least 20%, or even at least 30%. Nucleic acid molecules that reduce GLI2 mRNA and/or GLI2 protein levels by at least 40% (e.g., at least 50%, at least 60%, at least 70%, at least 75%, at least 80%, at least 85%, at least 90%, or even at least 95%) are particularly useful.

Nucleic acid molecules can be formulated into pharmaceutical compositions as described above and administered by any route, including, without limitation, oral or parenteral routes of administration such as intravenous, intramuscular, intraperitoneal, subcutaneous, intrathecal, intraarterial, nasal, transdermal (e.g., as a patch), or pulmonary absorption.

In some embodiments, methods of inhibiting tumor cell growth or treating cancer can include administering a chemotherapy agent or other drug used for treating a particular cancer as targeting GLI2 does not induce apoptosis. A chemotherapy agent can be administered before, after, or in combination with a nucleic acid molecule described herein. Non-limiting examples of chemotherapy agents include an alkylating agent such as a nitrogen mustard, ethylenimine or methylmelamine, alkyl sulfonate, nitrosourea, or triazene; an antimetabolite such as a folic acid analog, pyrimidine analog, or purine analog; a natural product such as a vinca alkaloid, epipodophyllotoxin, antibiotic, enzyme, or biological response modifier (e.g., interferon alpha or beta); platinum coordination complex; anthracendione; substituted urea; methylhydrazine derivative; adrenocortical suppressant (e.g., aminoglutethimide); hormones and antagonists such as adrenocorticosteroids, progestins, estrogens, antiestrogen, androgens, antiandrogen (e.g., Flutamide or Nilutamide), gonadotropin-releasing hormone analog (e.g., Leuprolide or Goserelin), or combinations thereof. Other non-limiting examples of drugs that are used for treating cancer include ketoconazole, which is used for treatment of prostate cancer, and Trastuzumab, a monoclonal antibody used for treatment of breast cancer.

Methods described herein can include monitoring the patient to, for example, determine if the cancer is improving with treatment. Any method can be used to monitor a patient. For example, for patients with prostate cancer, tumor size or levels of prostate specific antigen can be monitored. In addition, levels of GI2 protein or RNA can be monitored.

In some embodiments, nucleic acid molecules can be delivered by a gene therapy approach, using a nucleic acid construct from which siRNAs, shRNAs, ribozymes, or antisense oligonucleotides can be transcribed directly. For example, a nucleic acid construct encoding an RNA molecule that induces the degradation of RNA transcripts encoding GLI2 can be introduced into patients, tissue, or cells, and used to direct the expression of such an RNA molecule by exploiting the transcriptional machinery of the cell. Various gene therapy methods are well known in the art. See e.g., Kay et al., Nature Genet., 24:257-61 (2000); Cavazzana-Calvo et al., Science, 288:669 (2000); and Blaese et al., Science, 270: 475 (1995); Kantoff, et al., J. Exp. Med. 166:219 (1987). Any suitable gene therapy methods can be used including, e.g., those methods described in U.S. Patent Application Publication 2003/0148519.

In general, a nucleic acid encoding an siRNA, shRNA, enzymatic nucleic acid, or antisense oligonucleotide, and capable of inducing the degradation of RNA transcripts encoding GLI2, or otherwise specifically reducing cellular concentrations of GLI2, can be incorporated into an expression cassette or vector and operably linked to a promoter in the cassette or vector. Suitable promoters may be constitutive or inducible, and may be tissue or organ specific, or specific to a particular phase of development. Typically, the promoter is positioned 5′ to the region to be transcribed. Suitable promoters include but are not limited to viral transcription promoters derived from adenovirus, simian virus 40 (SV40) (e.g., the early and late promoters of SV40), Rous sarcoma virus (RSV), and cytomegalovirus (CMV) (e.g., CMV immediate-early promoter), human immunodeficiency virus (HIV) (e.g., long terminal repeat (LTR)), vaccinia virus (e.g., 7.5K promoter), and herpes simplex virus (HSV) (e.g., thymidine kinase promoter).

In one embodiment, the promoter is a U6 gene promoter or HII promoter. In another embodiment, the promoter is a promoter from a 7SL signal recognition particle RNA, or a 5S ribosomal RNA. In another embodiment, the promoter (for example, a U6 promoter) is modified so as to possess different specificity. As a non-limiting example, the U6 or HII promoter can be modified to a Tet-inducible promoter. In the Tet repressor, the presence of DNA-binding sites interferes with the initiation of transcription from the promoter. Thus, the presence of the Tet repressor at the TATA box, and other representative sequences, results in the U6 or HII promoter being repressed, or turned off. Addition of tetracycline results in the release of the Tet repressor, and concomitant de-repression of the promoter. Hence, the addition of tetracycline results in the induction, or “turning on,” of the modified promoter.

Other promoters are also contemplated and include other RNA polymerase III promoters, suitably modified as necessary. In addition to the U6 snRNA promoter, such promoters include tRNA, RNAse P RNA, and adenovirus VA RNA pol III promoters as described by Medina and Joshi (Curr. Opin. Mol. Ther. 1:580-594 (1999), Brummelcamp et al. (Science 296:550-553 (2002)), and McManus et al. (RNA 8:842-850 (2002)).

Where tissue-specific expression of the exogenous gene is desirable, tissue-specific promoters may be operably linked to the exogenous gene. In addition, selection markers may also be included in the vector for in vitro selection of cells that encode a nucleic acid molecule described herein. Various selection markers may be used including, e.g., genes conferring resistance to neomycin, hygromycin, zeocin, and combinations thereof.

In another aspect, DNA encoding an RNA molecule capable of inducing the degradation of RNA transcripts encoding GLI2 can be incorporated into a plasmid DNA vector. In one embodiment, a composition is provided that includes a vector having at least one expression cassette directing the expression of an RNA molecule capable of inducing the degradation of RNA transcripts encoding GLI2. The vectors may also encode marker genes, reporter genes, genes for selection of transformants or transfectants, or other genes of interest. Such vectors may also include specific sequences that allow for the stable integration of the vector-encoded expression cassettes into the genomes of host cells.

In some embodiments, an expression vector can include chromosomal, nonchromosomal, or synthetic DNA sequences, such as derivatives of viral DNAs. For example, viral DNAs can be from vaccinia, adenovirus, adeno-associated virus, fowl pox virus, pseudorabies, or retroviruses (e.g., lentiviruses). It is contemplated that any vector may be used as long as it is viable in the host cell, and adequately directs the expression of a nucleic acid molecule capable of inducing the degradation of RNA transcripts encoding GLI2. These criteria are sufficient for the vector to be used transiently transfect a host cell. However, vectors capable of replicating in the host cell, vectors that direct the stable integration of expression cassettes, or vectors that can otherwise be used to stably transfect host cells also can be used in some embodiments.

Many expression vectors that may be useful are commercially available, including, e.g., pSiren (BD Biosciences Clontech, Inc., Palo Alto, Calif.), pSilencer (Ambion, Inc., Austin, Tex.), pGE1 (Stratagene, Inc., La Jolla, Calif.), which are designed to direct the expression of shRNAs within host cells. In some embodiments, mammalian expression vectors include an origin of replication, suitable promoters and enhancers, as well as ribosome binding sites, polyadenylation sites, splice donor and acceptor sites, transcriptional termination sequences, and various 5′ and 3′ flanking non-transcribed sequences. Other exemplary vectors include, but are not limited to, the following eukaryotic expression vectors: pSG, pOG44, and pWLNEO (Stratagene, Inc., La Jolla, Calif.) and pSV2CAT (American Type Culture Collection). Particularly preferred vectors include pG1Na, a retroviral vector derived from MoMuLV (Zhou et al., Gene 149:3-39 (1994)); pCWRSV, an Adenovirus vector (Chaterjee et al., Science 258:1485 (1992)); pTZ18U (BioRad, Inc., Hercules, Calif.), and pLVTHM (Wiznerowicz and Trono, J. Virol. 77:8957-61 (2003)).

Various viral vectors may also be used. Typically, in a viral vector, the viral genome is engineered to eliminate the disease-causing capability of the virus, e.g., the ability to replicate in the host cells. Viral vectors are convenient to use as they can be easily introduced into cells, tissues and patients by way of infection. Once in the host cell, the recombinant virus typically is integrated into the genome of the host cell. In rare instances, the recombinant virus may also replicate and remain as extrachromosomal elements. Examples of preferred viral vectors that can be used to deliver one or more therapeutic nucleic acid molecules to cells or tissues are described in U.S. Patent Application Publication 2003/0138407.

A large number of retroviral vectors have been developed for gene therapy. These include vectors derived from oncoretroviruses (e.g., MLV), lentiviruses (e.g., HIV and SIV) and other retroviruses. For example, gene therapy vectors have been developed based on murine leukemia virus (See, Cepko, et al., Cell, 37:1053-1062 (1984), Cone and Mulligan, Proc. Natl. Acad. Sci. U.S.A., 81:6349-6353 (1984)), mouse mammary tumor virus (See, Salmons et al., Biochem. Biophys. Res. Commun., 159:1191-1198 (1984)), gibbon ape leukemia virus (See, Miller et al., J. Virology, 65:2220-2224 (1991)), HIV, (See Shimada et al., J. Clin. Invest., 88:1043-1047 (1991)), and avian retroviruses (See Cosset et al., J. Virology, 64:1070-1078 (1990)). In addition, various retroviral vectors are also described in U.S. Pat. Nos. 6,168,916; 6,140,111; 6,096,534; 5,985,655; 5,911,983; 4,980,286; and 4,868,116.

Adeno-associated virus (AAV) vectors also can be used. AAV vectors have been successfully tested in clinical trials. See e.g., Kay et al., Nature Genet. 24:257-61 (2000). AAV is a naturally occurring defective virus that requires other viruses such as adenoviruses or herpes viruses as helper viruses. See Muzyczka, Curr. Top. Microbiol. Immun., 158:97 (1992). A recombinant AAV virus useful as a gene therapy vector is disclosed in U.S. Pat. No. 6,153,436.

Adenoviral vectors can also be used for gene therapy. For example, U.S. Pat. No. 6,001,816 discloses an adenoviral vector which was used to deliver a leptin gene intravenously to a mammal to treat obesity. Other recombinant adenoviral vectors may also be used, including those disclosed in U.S. Pat. Nos. 6,171,855; 6,140,087; 6,063,622; 6,033,908; and 5,932,210.

Other useful viral vectors include recombinant hepatitis viral vectors (See, e.g., U.S. Pat. No. 5,981,274), and recombinant entomopox vectors (See, e.g., U.S. Pat. Nos. 5,721,352 and 5,753,258).

A nucleic acid molecule described herein can be introduced into cells or tissue in vitro or in a patient for purposes of gene therapy by various methods known in the art. For example, the nucleic acid molecule alone or in a conjugated or complex form described above, or incorporated into viral or DNA vectors, may be administered directly by injection into an appropriate tissue or organ of a patient. Alternatively, catheters or like devices, may be used to deliver exogenous gene sequences, complexes, or vectors into a target organ or tissue. Suitable catheters are disclosed in, e.g., U.S. Pat. Nos. 4,186,745; 5,397,307; 5,547,472; 5,674,192; and 6,129,705.

### Articles of Manufacture

Nucleic acid molecules described herein can be combined with packaging material and sold as a kit for treating cancer or inhibiting cancer cell growth. For example, shRNA molecules, vectors encoding shRNA molecules, ribozymes, or antisense oligonucleotides that inhibit expression of GLI2 can be combined with packaging material and sold as a kit for treating cancer or inhibiting cancer cell growth. Components and methods for producing articles of manufactures are well known. The articles of manufacture may combine one or more nucleic acid molecules (e.g., one or more shRNAs, ribozymes, or antisense oligonucleotides) as described herein. In addition, the articles of manufacture may further include reagents such as antibodies, buffers, indicator molecules, chemotherapy agents, and/or other useful reagents for treating cancer or inhibiting cancer cell growth. Instructions describing how the various reagents are effective for treating cancer or inhibiting cancer cell growth also may be included in such kits.

The invention will be further described in the following examples, which do not limit the scope of the invention described in the claims. All parts, ratios, percents and amounts stated in the Examples are by weight unless otherwise specified.

### EXAMPLES

### Example 1

### Materials and Methods

Tissue Culture and transfections: 293T human embryo kidney cells and prostate cancer cell lines 22Rv1, PC3, LnCap, DU145, and RWPEI were purchased from the American Type Culture Collection (ATCC). Prostate cancer cells and 293T cells were grown in DMEM-RPMI with 10% fetal bovine serum (FBS) and antibiotics at 37° C. and 5% CO₂. RWPE1 cells (non-tumorigenic prostate epithelial cells) were cultured in keratinocyte serum-free media containing bovine pituitary extract and epidermal growth factor (Invitrogen). RWPE1 stable clones were selected in complete medium containing G418 (Sigma-Aldrich) over 2 weeks. Transfections were done using the calcium phosphate procedure or lipofection with LipofectAMINE 2000 (Invitrogen).

Lentiviral-mediated shRNA delivery: For producing lentiviruses, 293T cells grown at 60% to 70% confluence were transfected with 10 μg of pLVTHM-Gli2 shRNA, 10 μg of pCMVΔR8.2, and 7.5 μg of pCMV-VSV-G plasmids by the calcium phosphate method. The next day (i.e., day 1), complete medium without antibiotics was used to replace the previous culture medium. The virus was collected as media supernatant on days 2 and 3, passed through a 0.45 μm filter, and stored at −80° C. At the time of transduction, 22Rv1 cells at 50% to 60% confluence were infected by adding 10 mL of medium containing lentiviruses twice at 12-h intervals, and then green fluorescent protein expression was analyzed 24 h after second infection.

Antibodies and Western blotting: Antibodies against Gli2(G20), β-actin, and horseradish peroxidase-conjugated secondary antibodies were purchased from Santa Cruz Biotechnology. Immunoblotting procedures were done as described by Bhatia et al. J. Biol. Chem. 281:19320-6 (2006).

Luciferase reporter assays: Prostate cells were transfected with 8×3′ Gli BS-LucII reporter, K17 luciferase reporter plasmid, or pGL3-Bcl2promo luciferase reporter plasmid, pSV40 β-gal (Promega), and respective shGLI2 expression plasmids. Luciferase and β-galactosidase activities were done using Luciferase Reporter Assay Reagent (Promega) and β-gal assay reagent (Pierce Biotechnology), respectively, according to the manufacturers' recommendations.

Colony formation assay: Scrambled and GLI2shRNAs were cotransfected with pTk-Puro in the indicated cell lines at a ratio of 19:1. The transfected cells were subcultured and selected with puromycin (Sigma; the final concentration of puromycin in the media varied for the cell lines from 1 to 3 μg/mL). Colonies that formed in 3 to weeks were fixed with 10% formalin for 2 h, washed with PBS, stained with 2% Gentian violet (Ricca Chemical Company) for 45 min, and air-dried.

Growth in soft agar: 22Rv1 prostate cancer cells were infected with lentiviruses encoding GLI2shRNA or scrambled shRNA. GLI2shRNA- and scrambled shRNA-expressing 22Rv1 cells were grown in six-well plates in complete media, trypsinized, washed with PBS, counted, and mixed at a concentration of 10,000 cells/2 mL of RPMI, 0.33% agar. The cells in 0.33% agar were plated over 2 mL of RPMI, 0.5% agar that had been allowed to harden in a six-well dish. Cells were fed 0.5 mL of RPMI twice a week and allowed to grow for 3 weeks. The cells were fixed with 10% formalin and stained with Gentian violet. Colony formation was determined under low magnification (×10) in an inverted microscope.

Xenograft growth: For the tumor growth studies, transduced 22Rv1 cells infected with lentiviruses encoding GLI2 shRNA or scrambled shRNA and BALB/c athymic (nude) male (NxGen Biosciences, Inc.) mice, 5 weeks old, were used. Cells (1×10⁶) in 0.1 mL of culture medium were mixed with 0.1 mL of Matrigel (BD Biosciences) and were injected subcutaneously (s.c.) into one flank of the mice. Tumors were measured and volume was calculated using the following formula: volume=½(4π/3) (L₁/2)(L₂/2)(H)=0.5238 L₁ L₂H, where L₁ is the long diameter, L₂ is the short diameter, and H is the height. The time to a target mean tumor volume of 500 mm³ is defined as the elapsed time from the date of cell implantation to the date when a 500-mm³ target is reached or when the mouse was sacrificed. Of the 15 mice in the study, 10 reached a target tumor volume of 500 mm³ by day 72, at which point experiments were concluded. A Kaplan-Meier survival analysis with the corresponding log-rank analysis was done using S-plus Software (Insightful). A linear regression analysis was used to measure the rate of mean tumor volume growth as a function of time using S-plus Software (Insightful). P<0.05 was considered to be statistically significant.

Proliferation assays: For measurement of proliferation, RWPE1 cells stably transfected with pcDNA3.1 or pcDNA3.1-Flag-Gli2cells were plated at 2×10³ per well into 96-well flat-bottomed microtiter plates (Falcon) in triplicate. Twenty microliters of CellTiter 96 AQueous One Solution Reagent (Promega) were added to each well of the assay plate containing the cells in 100 μl, of culture medium. The plate was incubated for 2 h at 37° C. in a humidified 5% CO₂ chamber. The absorbance was recorded at 490 nm using an ELISA reader. The proliferation was assessed 24, 48, and 72 h after the initial reading.

Cell cycle analysis. Cell cycle analysis was carried out in stable RWPE1 clones of pcDNA3.1+ (control) and cells expressing Gli2 by propidium iodide staining. Cells were synchronized by starving for growth factors and medium supplements for 24 h and harvested at different time points, 0, 6, 12, and 24 h, after adding the complete media to the cells. For fixing, 95% ethanol was added to cell suspension, incubated for 45 min at 4° C., and then stored at −20° C. overnight. Fixed cells were pelleted at 300×g for 10 min, washed twice with PBS and once with staining buffer (PBS, 2% FBS, and 0.01% NaN₃), and treated with RNase A (100 μg/mL) for 30 min at 37° C. Propidium iodide (25 μg/mL; Molecular Probes) staining was carried out at 37° C. for ≧30 min and stored at 4° C. until flow analysis.

### Example 2

### Identification of siRNA Target Sequences

siRNA sequences for the Gli2 target mRNA were selected using the siRNA Target Finder and Design Tool (Ambion, world wide web at ambion.com/techlib/misc/siRNA_finder.html). siRNAs were designed according to manufacturer instructions. Target siRNA sequences, along with the top and bottom strand oligonucleotide templates used to construct the shRNAs are shown in Table I.

The annealed shRNA insert was then cloned into a modified pLVTHM vector (see Wiznerowicz and Trono J. Virol. 2003, 77:8957-61).

TABLE I
Target Gli2 siRNA Sequences

| No. | Target Sequence | Top Strand Oligo | Bottom Strand Oligo |
|---|---|---|---|
| 1 | AAGGAAGGTACCATTACGAGC | 5′- | 5′- |
| | | CGCTCCCCGGAAGGTACCATTACG | CGATTTCCAAAAAGGAAGGTACCAT |
| | | AGCTTCAAGAGAGCTCGTAATGGT | TACGAGCTCTCTTGAAGCTCGTAAT |
| | | ACCTTCCTTTTTGGAAAT-3 ′ | GGTACCTTCCGGGGA-3′ |
| 2 | AAACCCTACATCTGCAAGATC | 5′- | 5′- |
| | | CGCGTCCCCACCCTACATCTGCAA | CGATTTCCAAAAAACCCTACATCTG |
| | | GATCTTCAAGAGAGATCTTGCAGA | CAAGATCTCTCTTGAAGATCTTGCA |
| | | TGTAGGGTTTTTTGGAAAT-3′ | GATGTAGGGTGGGGA-3′ |
| 3 | AAGATCTGGACAGGGATGACT | 5′- | 5′- |
| | | CGCGTCCCCGATCTGGACAGGGAT | CGATTTCCAAAAAGATCTGGACAGG |
| | | GACTTTCAAGAGAAGTCATCCCTG | GATGACTTCTCTTGAAAGTCATCCC |
| | | TCCAGATCTTTTTGGAAAT-3′ | TGTCCAGATCGGGGA-3′ |
| 4 | AAACACATGACCACCATGCAC | 5′- | 5′- |
| | | CGCGTCCCCACACATGACCACCAT | CGATTTCCAAAAAACACATGACCAC |
| | | GCACTTCAAGAGAGTGCATGGTGG | CATGCACTCTCTTGAAGTGCATGGT |
| | | TCATGTGTTTTTTGGAAAT-3′ | GGTCATGTGTGGGGA-3′ |
| 5 | AAATAACATGCCTGTGCAGTG | 5′- | 5′- |
| | | CGCGTCCCCATAACATGCCTGTGC | CGATTTCCAAAAAATAACATGCCTG |
| | | AGTGTTCAAGAGACACTGCACAGG | TGCAGTGTCTCTTGAACACTGCACA |
| | | CATGTTATTTTTTGGAAAT-3′ | GGCATGTTATGGGGA-3′ |

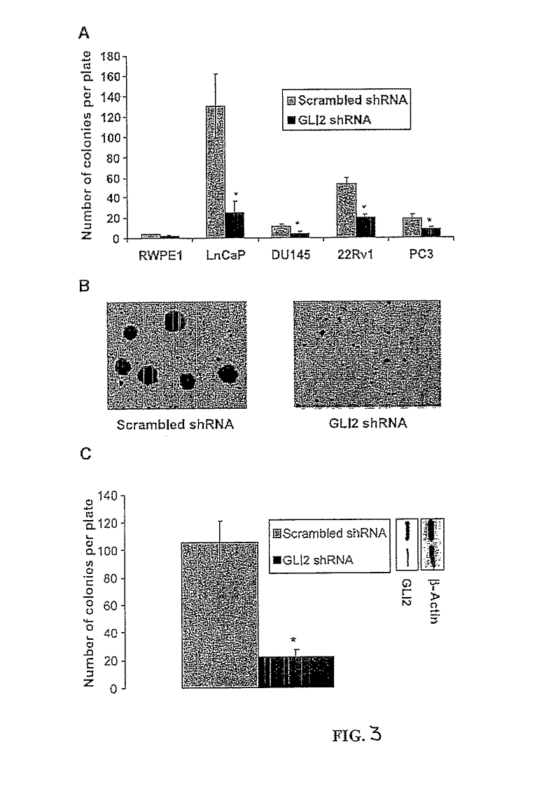

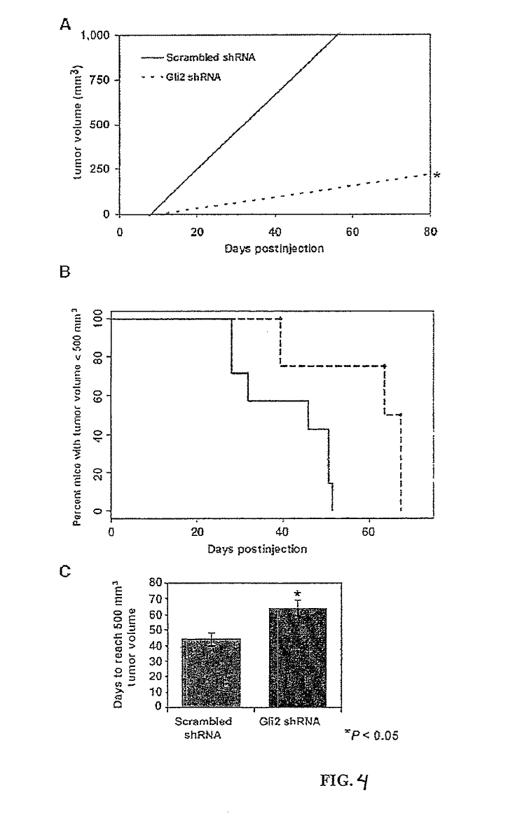

To determine which shRNA constructs were most effective at reducing expression of Gli2, 293T human embryo kidney cells were transfected with a control shRNA or one of the five targeted shRNAs shown in Table I, and GLI2 protein levels were examined by Western blotting (see FIG. 4). Cell lysates from 293T cells transfected with the Gli2 shRNA constructs were prepared and separated by SDS-PAGE (10% polyacrylamide) and then transferred to a nitrocellulose membrane in sample buffer (25 mM Tris, 190 mM glycine, and 20% methanol). The nitrocellulose membrane was incubated with G20, an antibody against GLI2, and horseradish peroxidase secondary antibodies (Santa Cruz Biotechnologies). Protein bands on the membrane were then visualized with a chemiluminescence reagent according to standard immunoblotting protocols, as described, for example, in Bhatia et al., 2006, J. Biol. Chem. 281: 19320-36. After detection of GLI2, the blot was stripped of anti-Gli2 antibody and then reprobed with an anti-β-actin antibody. Protein levels of Gli2 were quantified by densitometry.

The shRNA targeting the sequence GATCTGGACAGGGATGACT (SEQ ID NO:5) demonstrated the greatest reduction in GLI2 protein levels relative to GLI2 protein levels in the control samples. The shRNA targeting the sequence GGAAGGTACCATTACGAGC (SEQ ID NO:3) also reduced GLI2 protein levels. The remaining shRNA constructs targeting SEQ ID NOS: 4 (ACCCTACATCTGCAAGATC), 6 (ACACATGACCACCATGCAC), and 7 (ATAACATGCCTGTGCAGTG) were not as effective for reducing GLI2 protein levels. The shRNA construct targeting SEQ ID NO:5 was used in subsequent experiments.

### Example 3

### shRNA-Mediated Inhibition of GLI2 Inhibits GLI-Dependent Transcription in Prostate Cells

Prostate cancer cells demonstrate a high constitutive level of Hedgehog (Hh) signaling pathway activity and are dependent on GLI2 expression for transcription. To demonstrate that the shRNA construct targeting GLI2 reduces expression of GLI2 relative to control (i.e., non-tumorigenic RWPE1 cells), cell lysates were extracted from 293T cells transfected with either scrambled shRNA (control) or shRNA targeting GLI2. As seen in FIG. 5A, the shRNA construct targeting GLI2 showed a complete absence of GLI2 protein expression relative to control.

Prostate cancer cells were transfected with scrambled shRNA constructs and three different Gli-dependent luciferase reporter plasmids, and their luciferase activity was measured (and normalized to β-galactosidase activity). The measured luciferase activity is shown in FIGS. 5B-5D. Transfection with the Gli2 shRNA construct repressed activities of all three reporter constructs in prostate cancer cell lines LnCaP, DU145 and 22Rv1, which show a 3.2-, 3.0- and 4.1-fold inhibition of luciferase activity relative to cells transfected with the scrambled constructs. These data show that GLI2 plays a significant role in the pathway activation, and down-regulation of GLI2 is efficient in inhibiting transcriptional outcome of the Hh signaling pathway in prostate cancer cells.

### Example 4

### Knockdown of GLI2 Suppresses Proliferation and Anchorage-Independent Growth of Prostate Cancer Cells

Colony formation assay revealed that down-regulation of GLI2 leads to substantial inhibition of growth of all four human prostate cancer cell lines (DU145, PC3, 22Rr1, and LnCaP) examined as compared with scrambled shRNA control, whereas GLI2 knock-down in RWPE1 cells did not significantly affect the number of colonies (FIG. 6A).

To determine the effect of shRNA-mediated inhibition of GLI2 on anchorage-independent cell growth, 22Rv1 prostate cancer cells were infected with lentiviral constructs encoding scrambled shRNA or Gli2 shRNA as described in Example 1.

As shown in FIG. 6A-6C, colony formation is inhibited in all the prostate cancer cell lines transfected with the Gli2 shRNA construct, relative to cells transfected with scrambled shRNA, whereas no difference in colony formation is seen in the transfected RWPE1 cells relative to control, as shown in FIG. 6A.

Colony formation in soft agar was also inhibited 5-fold in 22Rv1 cells infected with a lentiviral Gli2 shRNA construct relative to the same cells infected with a lentiviral construct of scrambled shRNA, as seen in FIGS. 6B and 6C. These data show that knockdown of GLI2 dramatically inhibits anchorage-independent growth of 22Rr1 prostate cancer cells.

### Example 5

### Down-Regulation of GLI2 Inhibits Grown of 22Rv1 Xenografts

To investigate the role of Gli2 in the growth of prostate cancer cells in vivo, 22Rv1 prostate cancer cells were infected with lentiviral constructs of Gli2 shRNA or scrambled shRNA. The infected cells were then injected into nude mice and xenografts were obtained as described in Example 1.

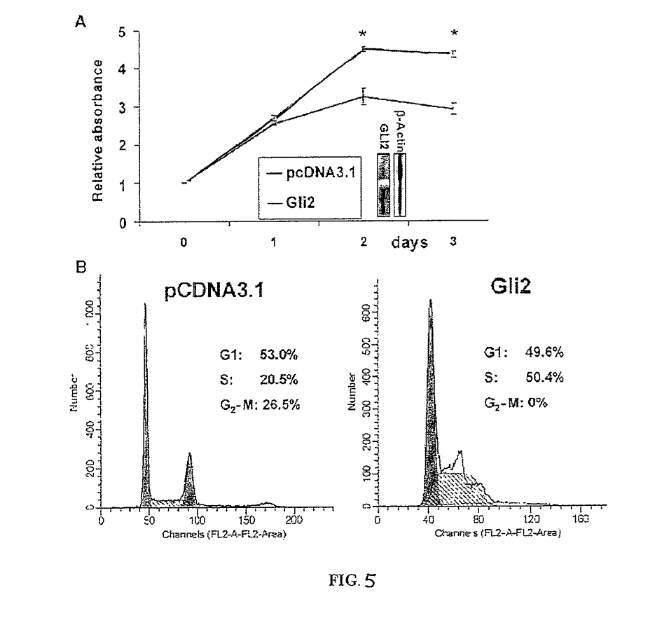

Linear regression analysis of the rate of tumor growth and multiplicity in the xenografts, as seen in FIGS. 7A-7C, shows that tumors expressing the Gli shRNA grew at a slower rate than tumors in xenografts expressing the scrambled shRNA construct (average, 3.1 and 20.7 mm³/d, respectively; FIG. 7A). The average time to a target tumor volume of 500 mm³ was also significantly longer in GLI2-shRNA-expressing than in scrambled shRNA-expressing xenografts (64 and 44 days, respectively; FIG. 7B). The observed differences were statistically significant, with P<0.05 according to a log-rank analysis (FIG. 7C). Together, these data indicate that inhibition of GLI2 is efficient in suppressing tumorigenic properties of prostate cancer cells both in vitro and in vivo.

### Example 6

### Overexpression of Gli2 Accelerates Growth of RWPE1 Cells

To assess GLI2 function in cells expressing GLI2 protein only at low levels, RWPE1 cells were stably transfected with pcDNA3.1 (as control) or pcDNA3.1-Flag-Gli2plasmids. RWPE1 cells that stably expressed Gli2 proliferated at a much faster rate than control transfected cells (FIG. 8A). Ectopic expression of Gli2 in RWPE1 cells resulted in both faster growth and higher saturation density. Cell cycle analysis revealed accumulation of these cells in S phase (˜50% cells transfected with Gli2, as compared with ˜20% cells transfected with empty vector) and almost complete disappearance of cells in G₂-M (FIG. 8B). These data indicate that ectopic expression of Gli2 accelerates cell cycle, especially transition through G₂-M, and stimulates proliferation of nontumorigenic prostate epithelial cells.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A nucleic acid molecule comprising a short hairpin ribonucleic acid (shRNA) comprising first and second regions that are each from 15 to 30 nucleotides in length, said first region comprising a nucleotide sequence that binds to the Gli2 target nucleotide sequence set forth in AAGATCTGGACAGGGATGACT (SEQ ID NO:5), and said second region having sufficient complementarity to said target nucleotide sequence to direct cleavage of a GLI2-encoding RNA transcript via RNA interference.

2-3. (canceled)

4. The nucleic acid molecule of claim 1, wherein said first and second regions occur within a single strand of RNA.

5. The nucleic acid of claim 4, wherein a linker region links said first and second regions.

6. The nucleic acid of claim 5, wherein said linker region comprises from 6 to 9 nucleotides.

7. The nucleic acid of claim 1, wherein said nucleic acid molecule is an intermolecular duplex, the first region occurring within a first strand of RNA and the second region occurring within a second strand of RNA.

8. The nucleic acid of claim 7, wherein the first and second regions of RNA each further comprise two 2′ deoxyribonucleotides at their 3′-ends.

9. A nucleic acid construct comprising a promoter operably linked to a nucleic acid molecule, said nucleic acid molecule comprising a short hairpin ribonucleic acid (shRNA) comprising first and second regions that are each from 15 to 30 nucleotides in length, said first region comprising the Gli2 target nucleotide sequence set forth in SEQ ID NO:5, and said second region having sufficient complementarity to said target nucleotide sequence for the nucleic acid molecule to direct cleavage of a GLI2-encoding RNA transcript via RNA interference.

10. A nucleic acid molecule comprising a short hairpin RNA (shRNA) comprising first and second regions that are each from 15 to 30 nucleotides in length, said first region corresponding to a target nucleotide sequence in the human Gli2 mRNA coding sequence set forth in SEQ ID NO: 3, 4, 5, 6, or 7; and said second region having sufficient complementarity to said target nucleotide sequence for the nucleic acid molecule to direct cleavage of a GLI2-encoding RNA transcript via RNA interference, wherein said first and second regions occur within a single strand of said nucleic acid and are linked by a linker region.

11. The nucleic acid molecule of claim 10, wherein said first region comprises from 19 to 30 consecutive nucleotides of SEQ ID NO: 1.

12-14. (canceled)

15. The nucleic acid of claim 10, wherein said linker region comprises from 6 to 9 nucleotides.

16. A nucleic acid construct comprising a promoter operably linked to a nucleic acid molecule, said nucleic acid molecule comprising a short hairpin RNA (shRNA) comprising first and second regions that are each from 15 to 30 nucleotides in length, said first region corresponding to a target nucleotide sequence in the human Gli2 mRNA coding sequence set forth in SEQ ID NO: 3, 4, 5, 6, or 7; and said second region having sufficient complementarity to said target nucleotide sequence for the nucleic acid molecule to direct cleavage of a GLI2-encoding RNA transcript via RNA interference, wherein said first and second regions occur within a single strand of said nucleic acid and are linked by a linker region.

17. The nucleic acid construct of claim 16, wherein said promoter is a HII promoter.

18. A composition comprising the nucleic acid of claim 1 or claim 10 and a pharmaceutically acceptable excipient, said composition decreasing the level of a Gli2 mRNA or polypeptide when introduced into a cell.

19. A method of inhibiting tumor cell growth in an individual, the method comprising administering to an individual a composition that inhibits expression of Gli2 by RNA interference, the composition comprising the nucleic acid molecule of claim 1.

20. The method of claim 19, wherein the method comprises inhibiting prostate tumor cell growth.

21. The method of claim 19, wherein the composition that inhibits expression of Gli2 further comprises a pharmaceutically acceptable excipient.

22. A method of treating cancer, comprising administering to an individual a composition that inhibits expression of Gli2, the composition comprising the nucleic acid molecule of claim 9.

23. The method of claim 22, said method further comprising administering a chemotherapeutic agent.

24. The method of claim 23, wherein the chemotherapeutic agent is an agent capable of causing apoptosis of cancer cells.

25. The method of claim 22, wherein the cancer is selected from the group consisting of prostate cancer, basal cell carcinoma, medulloblastoma, pancreatic cancer, gastric cancer, hepatocellular carcinoma, breast cancer, and lung cancer.

26. The method of claim 22, wherein the composition that inhibits expression of Gli2 further comprises a pharmaceutically acceptable excipient.

27. A method of treating cancer, comprising administering to an individual a composition that inhibits expression of Gli2, the composition comprising the nucleic acid molecule of claim 10 and a pharmaceutically acceptable excipient.


SEQUENCE LISTING (7 sequences)

SEQ ID NO: 1 - DNA, 6780 bp, Homo sapiens
gattgccacccaggacgatgagcggctgagatggagacgtctgcctcagccactgcctccgagaagcaagaagccaaaagtgggatcctggaggccgctggcttccccgacccgggtaaaaaggcctctcctttggtggtggctgcagcggcagcagcagcggtagctgcccaaggagtgccgcagcatctcttgccaccattccatgcgcccctaccgattgacatgcgacaccaggaaggaaggtaccattacgagcctcattctgtccacggtgtgcacgggccccctgccctcagcggcagccctgtcatctctgacatctccttgatccggctttccccgcacccggctggccctggggagtcccccttcaacgccccccacccgtacgtgaacccccacatggagcactacctccgttctgtgcacagcagccccacgctctccatgatctctgcagccaggggcctcagccccgctgatgtggcccaggagcaccttaaggagaggggactgtttggccttcctgctccaggcaccaccccctcagactattaccaccagatgaccctcgtggcaggccaccccgcgccctacggggacctgctgatgcagagcgggggcgctgccagcgcaccccatctccacgactacctcaaccccgtggacgtgtcccgtttctccagcccgcgggtgacgccccgcctgagccgcaagcgggcgctgtccatctccccactctcagacgccagcctggacctgcagcggatgatccgcacctcacccaactcgctagtggcctacatcaacaactcccgaagcagctcggcggccagcggttcctacgggcatctgtcagcgggtgccctcagcccagccttcaccttcccccaccccatcaaccccgtggcctaccagcagattctgagccagcagaggggtctggggtcagcctttggacacacaccacccctgatccagccctcacccaccttcctggcccagcagcccatggccctcacctccatcaatgccacgcccacccagctcagcagcagcagcaactgtctgagtgacaccaaccagaacaagcagagcagtgagtcggccgtcagcagcaccgtcaaccctgtcgccattcacaagcgcagcaaggtcaagaccgagcctgagggcctgcggccggcctcccctctggcgctgacgcagggccaggtgtctggacacggctcatgtgggtgtgcccttcccctctcccaggagcagctggctgacctcaaggaagatctggacagggatgactgtaagcaggaggctgaggtggtcatctatgagaccaactgccactgggaagactgcaccaaggagtacgacacccaggagcagctggtgcatcacatcaacaacgagcacatccacggggagaagaaggagtttgtgtgccgctggcaggcctgcacgcgggagcagaagcccttcaaggcgcagtacatgctggtggtgcacatgcggcgacacacgggcgagaagccccacaagtgcacgttcgagggctgctcgaaggcctactcccgcctggagaacctgaagacacacctgcggtcccacaccggggagaagccatatgtgtgtgagcacgagggctgcaacaaagccttctccaacgcctcggaccgcgccaagcaccagaatcgcacccactccaacgagaaaccctacatctgcaagatcccaggctgcaccaagagatacacagaccccagctctctccggaagcatgtgaaaacggtccacggcccagatgcccacgtcaccaagaagcagcgcaatgacgtgcacctccgcacaccgctgctcaaagagaatggggacagtgaggccggcacggagcctggcggcccagagagcaccgaggccagcagcaccagccaggccgtggaggactgcctgcacgtcagagccatcaagaccgagagctccgggctgtgtcagtccagccccggggcccagtcgtcctgcagcagcgagccctctcctctgggcagtgcccccaacaatgacagtggcgtggagatgccggggacggggcccgggagcctgggagacctgacggcactggatgacacacccccaggggccgacacctcagccctggctgccccctccgctggtggcctccagctgcgcaaacacatgaccaccatgcaccggttcgagcagctcaagaaggagaagctcaagtcactcaaggattcctgctcatgggccgggccgactccacacacgcggaacaccaagctgcctcccctcccgggaagtggctccatcctggaaaacttcagtggcagtgggggcggcgggcccgcggggctgctgccgaacccgcggctgtcggagctgtccgcgagcgaggtgaccatgctgagccagctgcaggagcgccgcgacagctccaccagcacggtcagctcggcctacaccgtgagccgccgctcctccggcatctccccctacttctccagccgccgctccagcgaggcctcgcccctgggcgccggccgcccgcacaacgcgagctccgctgactcctacgaccccatctccacggacgcgtcgcggcgctcgagcgaggccagccagtgcagcggcggctccgggctgctcaacctcacgccggcgcagcagtacagcctgcgggccaagtacgcggcagccactggcggccccccgcccactccgctgccgggcctggagcgcatgagcctgcggaccaggctggcgctgctggacgcgcccgagcgcacgctgcccgccggctgcccacgcccactggggccgcggcgtggcagcgacgggccgacctatggccacggccacgcgggggctgcgcccgccttcccccacgaggctccaggcggcggagccaggcgggccagcgaccctgtgcggcggcccgatgccctgtccctgccgcgggtgcagcgcttccacagcacccacaacgtgaaccccggcccgctgccgccctgtgccgacaggcgaggcctccgcctgcagagccacccgagcaccgacggcggcctggcccgcggcgcctactcgccccggccgcctagcatcagcgagaacgtggcgatggaggccgtggcggcaggagtggacggcgcggggcccgaggccgacctggggctgccggaggacgacctggtgcttccagacgacgtggtgcagtacatcaaggcgcacgccagtggcgctctggacgagggcaccgggcaggtgtatcccacggaaagcactggcttctctgacaaccccagactacccagcccggggctgcacggccagcgcaggatggtggctgcggactccaacgtgggcccctccgcccctatgctgggaggatgccagttaggctttggggcgccctccagcctgaacaaaaataacatgcctgtgcagtggaatgaggtgagctccggcaccgtagacgccctggccagccaggtgaagcctccaccctttcctcagggcaacctggcggtggtgcagcagaagcctgcctttggccagtacccgggctacagtccgcaaggcctacaggctagccctgggggcctggacagcacgcagccacacctgcagccccgcagcggagccccctcccagggcatccccagggtaaactacatgcagcagctgcgacagccagtggcaggcagccagtgtcctggcatgactaccactatgagcccccatgcctgctatggccaagtccacccccagctgagccccagcaccatcagtggggccctcaaccagttcccccaatcctgcagcaacatgccagccaagccagggcatctggggcaccctcagcagacagaagtggcacctgaccccaccacgatgggcaatcgccacagggaacttggggtccccgattcagccctggctggagtgccaccacctcacccagtccagagctacccacagcagagccatcacctggcagcctccatgagccaggagggctaccaccaggtccccagccttctgcctgcccgccagcctggcttcatggagccccaaacaggcccgatgggggtggctacagcaggctttggcctagtgcagccccggcctcccctcgagcccagccccactggccgccaccgtggggtacgtgctgtgcagcagcagctggcctacgccagggccacaggccatgccatggctgccatgccgtccagtcaggaaacagcagaggctgtgcccaagggagcgatgggcaacatggggtcggtgcctccccagccgcctccgcaggacgcaggtggggccccggaccacagcatgctctactactacggccagatccacatgtacgaacaggatggaggcctggagaacctcgggagctgccaggtcatgcggtcccagccaccacagccacaggcctgtcaggacagcatccagccccagcccttgccctcaccaggggtcaaccaggtgtccagcactgtggactcccagctcctggaggccccccagattgacttcgatgccatcatggatgatggcgatcactcgagtttgttctcgggtgctctgagccccagcctcctccacagcctctcccagaactcctcccgcctcaccaccccccgaaactccttgaccctgccctccatccccgcaggcatcagcaacatggctgtcggggacatgagctccatgctcaccagcctcgccgaggagagcaagttcctgaacatgatgacctagaggcccgagcgcctggtgctgagtgcacccggaggggtcatcgctgcccagagcctggggattccagctgtcttgtctttttccaaaaaagtgttaaataggcttgaggggttgttgcgcaatggccgcttcagatgacagatgttgtaagagaaggtttatgggcatcctctctggtcttttggattattcctcagaacaatgaaaaaagtctccataggacaggaaggaatgcaaaactcatttacacagtgctttccagcctttggtgcttacaggaccgcgctgttccggcttcttcacggctgacattcggctaacgagggattactttggccaaaacctttcaaaggatatgcagaaagatggtagggagcatttgggtttgaatctgaatgctatactggatactctgctccggaaagatgagctttttattctactacttggaaggaaaaggaattcctggtccacctgaattcctctatgaagcctaactcttgaggtctctaacataccttgtcatagaggaaaagcacagattatacctggatgattcaggagcacattctgattccaggtttggtagagctggctcttctactccgtaaagccgagtctgggactggcagcccatccaagtgtatatgaatgaataaagcatccaagtatatatgaatgaataaagtatgtaagtatcaccagaaaaaggaaagaaaaaatgtactccttggggcaagcccagaagctgccctggcctctccagaccgtgtttacagtgtttgcatgtagaatgtagcccttcctgaaaagaagacttgtttctaaatacctcggggctgctggagccgctgtgggttagggatggactgaggcctcgaggagtgagggtgcacccggggcccagcctcaggctgccctagggatctctcagtaggaagaggaagttgcgtgtttacccaatcctgtttctccaatgcaacgtccacccactttaccaccaaaaactccagggcctgacggcagcccggtcccccagcactcaccagcagcccagtgttctccaccaagccacagtgtgcatgcctggtatcctccggattcccttccttctgcccgctgagtcactgggcagagaatgatgacatgtgtaggtggtgtggttgggggtggaaaggggaaggggttgatcctcaggactctgagggagcatcgttgaattttcctgttcagtgtgaccaagacccacctggaaatggaatttggaactggcttcaggagacatcattcctgaacacactgtagggtgaattggtgcatcttccccaccatacacacacacacacacacacacacacacacacacacacacacaccccaaaccttttcatggggaatgtgtggcaaccttgccaaacagcaccactcagagtgtgactctgactgtgaccttggccttaatgaggaacttcttaggagagtttgaggacaaggccaacatcgtcatctgggctcgctgcgtcccagcacatcaaactctgtccagagacaaggccaactgcaaatgaaagccagggaacattgctaagggtctgtggctctgtggtggtgttcatcgccttcctgagataggatttcccttgccagtcccaacctgtatatattctgtacagaagacatccctgaatatactgtaggtgagtcgtccagccaaatttatatctccaaaacatttttagctttttctacatgctatgaattgagatgacatgctcaacttgtaaataagtctttttgtacattaaaaaagtaattttttcataatttatcttgtctatctgcttcccccttgacagtagttaatgagaacctgggcagtaaatttggtgcattcgagcagaaattaggctgtattttttcttaacagtgtcaaaattgactatcccgcctttgccaagaaatgtttaatgctgaggcaaaaaaaaaaaa

SEQ ID NO: 2 - protein, 1560 aa, Homo sapiens
METSASATASEKQEAKSGILEAAGFPDPGKKASPLVVAAAAAAAVAAQGVPQHLLPPFHAPLPIDMRHQEGRYHYEPHSVHGVHGPPALSGSPVISDISLIRLSPHPAGPGESPFNAPHPYVNPHMEHYLRSVHSSPTLSMISAARGLSPADVAQEHLKERGLFGLPAPGTTPSDYYHQMTLVAGHPAPYGDLLMQSGGAASAPHLHDYLNPVDVSRFSSPRVTPRLSRKRALSISPLSDASLDLQRMIRTSPNSLVAYINNSRSSSAASGSYGHLSAGALSPAFTFPHPINPVAYQQILSQQRGLGSAFGHTPPLIQPSPTFLAQQPMALTSINATPTQLSSSSNCLSDTNQNKQSSESAVSSTVNPVAIHKRSKVKTEPEGLRPASPLALTQGQVSGHGSCGCALPLSQEQLADLKEDLDRDDCKQEAEVVIYETNCHWEDCTKEYDTQEQLVHHINNEHIHGEKKEFVCRWQACTREQKPFKAQYMLVVHMRRHTGEKPHKCTFEGCSKAYSRLENLKTHLRSHTGEKPYVCEHEGCNKAFSNASDRAKHQNRTHSNEKPYICKIPGCTKRYTDPSSLRKHVKTVHGPDAHVTKKQRNDVHLRTPLLKENGDSEAGTEPGGPESTEASSTSQAVEDCLHVRAIKTESSGLCQSSPGAQSSCSSEPSPLGSAPNNDSGVEMPGTGPGSLGDLTALDDTPPGADTSALAAPSAGGLQLRKHMTTMHRFEQLKKEKLKSLKDSCSWAGPTPHTRNTKLPPLPGSGSILENFSGSGGGGPAGLLPNPRLSELSASEVTMLSQLQERRDSSTSTVSSAYTVSRRSSGISPYFSSRRSSEASPLGAGRPHNASSADSYDPISTDASRRSSEASQCSGGSGLLNLTPAQQYSLRAKYAAATGGPPPTPLPGLERMSLRTRLALLDAPERTLPAGCPRPLGPRRGSDGPTYGHGHAGAAPAFPHEAPGGGARRASDPVRRPDALSLPRVQRFHSTHNVNPGPLPPCADRRGLRLQSHPSTDGGLARGAYSPRPPSISENVAMEAVAAGVDGAGPEADLGLPEDDLVLPDDVVQYIKAHASGALDEGTGQVYPTESTGFSDNPRLPSPGLHGQRRMVAADSNVGPSAPMLGGCQLGFGAPSSLNKNNMPVQWNEVSSGTVDALASQVKPPPFPQGNLAVVQQKPAFGQYPGYSPQGLQASPGGLDSTQPHLQPRSGAPSQGIPRVNYMQQLRQPVAGSQCPGMTTTMSPHACYGQVHPQLSPSTISGALNQFPQSCSNMPAKPGHLGHPQQTEVAPDPTTMGNRHRELGVPDSALAGVPPPHPVQSYPQQSHHLAASMSQEGYHQVPSLLPARQPGFMEPQTGPMGVATAGFGLVQPRPPLEPSPTGRHRGVRAVQQQLAYARATGHAMAAMPSSQETAEAVPKGAMGNMGSVPPQPPPQDAGGAPDHSMLYYYGQIHMYEQDGGLENLGSCQVMRSQPPQPQACQDSIQPQPLPSPGVNQVSSTVDSQLLEAPQIDFDAIMDDGDHSSLFSGALSPSLLHSLSQNSSRLTTPRNSLTLPSIPAGI

SEQ ID NO: 3 - DNA, 21 bp, Homo sapiens
aaggaaggtaccattacgagc

SEQ ID NO: 4 - DNA, 21 bp, Homo sapiens
aaaccctacatctgcaagatc

SEQ ID NO: 5 - DNA, 21 bp, Homo sapiens
aagatctggacagggatgact

SEQ ID NO: 6 - DNA, 21 bp, Homo sapiens
aaacacatgaccaccatgcac

SEQ ID NO: 7 - DNA, 21 bp, Homo sapiens
aaataacatgcctgtgcagtg